(19) United States
(12) Patent Application Publication
(10) Pub. No.: US 2026/0345041 A1
(43) Pub. Date: Nov. 5, 2026

(54) Gas Backfill of Vacutainer Tubes and Packaging to Improve Blood Gas and Shelf Life Performance

(71) Applicant: Becton, Dickinson and company, Franklin Lakes, NJ (US)

(72) Inventors: Brian Chen Syverud, Glen Rock, NJ (US); Yosang Yoon, Edgewater, NJ (US); Daniel Feltyberger, Sparta, NJ (US); Arun U. Nair, Denville, NJ (US); Adam Edelhauser, Kinnelon, NJ (US); George Emad Youssef, Holmdel, NJ (US); Hocheul Kim, Kearny, NJ (US)

(21) Appl. No.: 19/118,026

(22) PCT Filed: Oct. 4, 2023
(86) PCT No.: PCT/US2023/075917

§ 371 Date: Apr. 3, 2025

(51) Int. Cl. A61B 5/15

(52) U.S. Cl. CPC A61B 5/150274; A61B 5/150351

(57) ABSTRACT

A biological liquid collection device including a collection module for receiving a biological liquid sample, an evacuated container having an open end and a closed end, said evacuated container containing the collection module therein, and a closure for closing the open end of the evacuated container, wherein the evacuated container comprises a gas composition with select partial pressure of a targeted gas that is substantially greater than a targeted gas partial pressure of atmosphere external to an internal cavity of the evacuated container.

### CROSS-REFERENCE TO RELATED APPLICATIONS

The present application claims priority to both U.S. Provisional Patent Application No. 63/413,013 entitled “Gas Backfill of Vacutainer Tubes to Decrease Rate of Draw Volume Loss and Improve Shelf Life Performance” filed Oct. 4, 2022, and U.S. Provisional Patent Application No. 63/510,476 entitled “Gas Backfill of Vacutainer Tubes to Decrease Rate of Draw Volume Loss and Improve Shelf Life Performance” filed Jun. 27, 2023, the entirety of both applications is hereby incorporated by reference in their entireties.

### BACKGROUND OF THE INVENTION

### Field of the Invention

The disclosure relates, in general, to a collection device and a method of making a balanced fluid collection device for collecting a biological fluid sample, and more particularly, to a blood sample collection device integrated with an evacuated blood collection tube for use in connection with blood gas analysis and even more particularly, to a blood sample collection device designed to draw blood using an “atmospheric-balanced vacuum” to ensure the blood is exposed to the sample atmospheric partial pressure oxygen and partial pressure carbon dioxide levels as found in a standard arterial blood gas (ABG) syringe, resulting in blood gas sample stabilization during collection.

### Background of the Related Art

A 1 mL-3 mL syringe-based platform is commonly accepted for blood gas laboratory tests. Current blood gas devices fall within two categories based on the filling methods employed (1) plunger-user assisted and (2) vented-blood pressure assisted. These syringe configurations typically require the user to follow a protocol that involves air purging, capping/sealing, and anticoagulant mixing steps to ensure blood sample quality isn't compromised for analysis in the diagnostic instruments. Besides the complicated multistep workflow, conventional blood collection syringes significantly elevate the safety risk for blood exposure during the air burp and capping procedure.

A recent device for blood collection for collecting small samples of blood and dispensing a portion of the sample into a device intended or designed to analyze the sample, such as point-of-care or a near-patient testing device, is disclosed in U.S. Pat. No. 9,649,061, the entirety of which is incorporated herein by reference. The blood sample collection device disclosed therein is integrated within an evacuated container, such as a BD Vacutainer® blood collection tube, owned by Becton, Dickinson, and Company, the assignee of the present invention. Use of this device allows for blood sample collection and dispensing for point-of-care applications which incorporates conventional automatic blood draw and includes a novel controlled sample dispensing capability while minimizing exposure risk. When blood fills a conventional Vacutainer® tube, the gas composition, dissolved and bound to hemoglobin in the blood (O₂, N₂, CO₂), is exposed to a gas mixture in the tube where each respective gas mixture component has its own partial pressure. The total pressure in the tube is the sum of the partial pressure of each individual gas (Ptube=PO₂+PCO₂+PN₂) as demonstrated by Dalton's law of partial pressures. This fundamental property of gases dictates a traditional tube vacuum pressure of 300 mmHg. However, the internal pressure of a tube is defined by an internal volume of the tube and a desired draw volume of the tube (e.g., 1 mL, 2 mL, etc.). In comparison, normal atmospheric gas composition has an oxygen partial pressure of 160 mmHg at atmospheric pressure and 760 mmHg at sea level. This standard vacuum process creates an environment that exposes blood to a larger partial pressure gradient (ΔP) for both oxygen and carbon dioxide in a conventional Vacutainer® tube in comparison to a syringe that can then lead to blood gas bias. As a result, gases can come out of solution (blood), as determined by the equilibrium between the undissolved gas in the vacuum tube and the gas dissolved in the blood.

Although air is a mix of approximately 80% Nitrogen and approximately 20% Oxygen, the two gases permeate into an evacuated tube independently. Oxygen permeates approximately ten times faster than Nitrogen and is responsible for the majority of tube draw volume loss over the shelf life of an evacuated container.

There is a need in the art for an atmospheric-balanced vacuum tube architecture that reduces blood gas bias and enables stable blood gas levels during blood vacuum draws using conventional blood collection devices. There is also a need in the art for an atmospheric-balanced vacuum tube architecture that provides a superior vacuum shelf-life by reducing the gas permeation rate through the plastic tube. There is a further need in the art for an atmospheric-balanced conventional specimen collection container, such as an evacuated blood collection tube, that provides a superior vacuum shelf-life by reducing the gas permeating rate through the material plastic.

### SUMMARY OF THE INVENTION

The key benefits of the arterial blood gas (ABG) atmospheric-balanced vacuum tube of the present disclosure is the reduction in both blood collection workflow steps and blood exposure associated with conventional (ABG) syringe blood collection sets. The device of the present disclosure provides a simplified user workflow as it uses a vacuum drawing method to uniformly mix anticoagulant in a fixed maximum blood sample that is air free. A plug element is located at a fixed position in a tip cap. This plug element is air permeable and liquid impermeable to allow air to be purged as the device fills and subsequently seals off upon blood contact. This atmospheric-balanced vacuum design of the present disclosure allows the removal of a dispenser component from the evacuated tube, which allows a controlled sample to be dispensed to a diagnostic instrument cartridge or aspiration by/through a probe in a blood gas diagnostic port.

According to one aspect, a biological liquid collection device may include a collection module for receiving a biological liquid sample; an evacuated container having an open end and a closed end, said evacuated container containing the collection module therein; and a closure for closing the open end of the evacuated container, wherein the evacuated container comprises a gas composition with select partial pressure of a targeted gas that is substantially greater than a targeted gas partial pressure of atmosphere external to an internal cavity of the evacuated container.

According to another aspect, the evacuated container may include the gas composition with select partial pressures of targeted gasses that are greater than the targeted gas partial pressures of the atmosphere outside of the evacuated container. The gas composition within the evacuated container may include oxygen, carbon dioxide, and nitrogen. The oxygen in the gas composition located within the evacuated container may have a partial pressure that is greater than a partial pressure of atmospheric oxygen outside of the evacuated container. The carbon dioxide in the gas composition located within the evacuated container may have a partial pressure that is substantially equal to a partial pressure of atmospheric carbon dioxide outside of the evacuated container. The gas composition may include approximately 75% oxygen, approximately 23% nitrogen, and approximately 0.1% carbon dioxide. The evacuated container may have a total pressure of 300 mmHg and wherein the oxygen within the gas composition in the evacuated container has a partial pressure of approximately 160 mmHg. The evacuated container may have a total pressure of 300 mmHg and wherein the carbon dioxide within the gas composition in the evacuated container has a partial pressure of approximately 0.3 mmHg. The oxygen within the gas composition of outside air may have a partial pressure of approximately 160 mmHg and the carbon dioxide within the gas composition of the outside air may have a partial pressure of approximately 0.3 mmHg. The collection module may include a first end having a sample introduction opening, a second end having a sample dispensing opening, a passageway extending between the sample introduction opening and the sample dispensing opening, and a porous plug covering the second end of a housing. A closure may be configured to close the sample introduction opening in the collection module and wherein the closure comprises a pierceable self-sealing stopper. The porous plug may be adapted to allow air to pass from the passageway of the collection module while preventing the biological liquid sample to pass therethrough. When the evacuated container is one of a 1 mL 13×75 tube, 2 mL 13×75 tube, 2.5 mL 13×75 tube, 3.5 mL 13×75 tube, and 4 mL 13×75 tube, the evacuated container may have a shelf life of at least 10 months, 24 months, 24 months, 12 months, and 21 months, respectively. The gas composition in the evacuated container may include argon. The gas composition within the evacuated container may include oxygen, nitrogen, and a third gas such as Argon with a similar permeation rate to nitrogen. The oxygen in the gas composition located within the evacuated container may have a partial pressure that is equal to the partial pressure of atmospheric oxygen outside of the evacuated container, and the Argon in the gas composition located within the evacuated container has a partial pressure that is greater than the partial pressure of atmospheric Argon outside of the evacuated container. The evacuated container may be a partial draw tube.

According to one aspect, a biological liquid collection device may include a collection module for receiving a biological liquid sample; an evacuated container containing the collection module therein; and a closure for closing an open end of the evacuated container, wherein the evacuated container comprises a gas composition that has an enriched oxygen content having a partial pressure substantially greater than a partial pressure of oxygen in air at atmospheric pressure of 760 mmHg external to an inner cavity of the evacuated container.

According to one aspect, the evacuated container may have a partial pressure of approximately 300 mmHg and wherein the partial pressure of oxygen within the evacuated container is approximately 160 mmHg. The gas composition may include carbon dioxide and nitrogen, and wherein the partial pressure of carbon dioxide within the evacuated container may be approximately 0.3 mmHg and the partial pressure of nitrogen within the evacuated container may be approximately 140 mmHg. The evacuated container may have a partial pressure of approximately 300 mmHg and wherein the partial pressure of oxygen within the evacuated container is greater than 160 mmHg. The gas composition may include approximately 75% oxygen. The gas composition may include approximately 23% nitrogen and approximately 0.1% carbon dioxide.

According to one aspect, a method of making an atmospheric balanced fluid collection device may include providing a container having an open end and a closed end, said container defining a chamber; drawing a vacuum within the container to remove at least some gas from within the chamber; back purging the chamber with a gas composition that is proportioned greater than a gas composition of atmosphere outside of an evacuated container, wherein the back purging of the chamber is conducted until reaching a predetermined vacuum pressure within the container; drawing a further vacuum within the container to remove at least some of the gas from within the chamber; back purging the chamber with a further gas composition that is proportioned greater than the gas composition of the atmosphere outside of the evacuated container, wherein the back purging of the chamber is conducted until reaching the predetermined vacuum pressure within the container; and closing the open end of the container.

According to one aspect, the predetermined vacuum pressure within the container may be 300 mmHg and the gas composition may include approximately 75% oxygen having a partial pressure of approximately 160 mmHg. The method may further include placing a fluid collection module within the container, wherein the fluid collection module comprises a first end having a sample introduction opening, a second end having a sample dispensing opening, a passageway extending between the sample introduction opening and the sample dispensing opening, and a porous plug covering the second end of a housing, said porous plug being adapted to allow air to pass from the passageway of the collection module while preventing a biological liquid sample to pass therethrough. When the evacuated container is one of a 1 mL 13×75 tube, 2 mL 13×75 tube, 2.5 mL 13×75 tube, 3.5 mL 13×75 tube, and 4 mL 13×75 tube, the evacuated container may have a shelf life of at least 10 months, 24 months, 24 months, 12 months, and 21 months, respectively.

According to one aspect, a biological liquid collection device assembly may include an evacuated tube for receiving a biological liquid sample; and a barrier packaging, said barrier packaging containing the evacuated tube therein, wherein the barrier packaging comprises a gas composition with select partial pressure of a targeted gas that is substantially greater than a targeted gas partial pressure of atmosphere external to the barrier packaging. The evacuated tube may also include a gas composition with select partial pressure of a targeted gas that is substantially greater than a targeted gas partial pressure of atmosphere external to the barrier packaging.

The present invention is also disclosed in the following clauses:
Clause 1: A biological liquid collection device, comprising a collection module for receiving a biological liquid sample; an evacuated container having an open end and a closed end, said evacuated container containing the collection module therein; and a closure for closing the open end of the evacuated container, wherein the evacuated container comprises a gas composition with select partial pressure of a targeted gas that is substantially greater than a targeted gas partial pressure of atmosphere external to an internal cavity of the evacuated container.
Clause 2: The biological liquid collection device of Clause 1, wherein the evacuated container comprises the gas composition with select partial pressures of targeted gasses that are greater than the targeted gas partial pressures of the atmosphere outside of the evacuated container.
Clause 3: The biological liquid collection device of Clause 1 or Clause 2, wherein the gas composition within the evacuated container comprises oxygen, carbon dioxide, and nitrogen.
Clause 4: The biological liquid collection device of Clause 3, wherein the oxygen in the gas composition located within the evacuated container has a partial pressure that is greater than a partial pressure of atmospheric oxygen outside of the evacuated container.
Clause 5: The biological liquid collection device of Clause 3 or Clause 4, wherein the carbon dioxide in the gas composition located within the evacuated container has a partial pressure that is substantially equal to a partial pressure of atmospheric carbon dioxide outside of the evacuated container.
Clause 6: The biological liquid collection device of any of Clauses 3-5, wherein the gas composition comprises approximately 75% oxygen, approximately 23% nitrogen, and approximately 0.1% carbon dioxide.
Clause 7: The biological liquid collection device of Clause 6, wherein the evacuated container has a total pressure of 300 mmHg and wherein the oxygen within the gas composition in the evacuated container has a partial pressure of approximately 160 mmHg.
Clause 8: The biological liquid collection device of Clause 7, wherein the evacuated container has a total pressure of 300 mmHg and wherein the carbon dioxide within the gas composition in the evacuated container has a partial pressure of approximately 0.3 mmHg.
Clause 9: The biological liquid collection device of Clause 8, wherein the oxygen within the gas composition of outside air has a partial pressure of approximately 160 mmHg and the carbon dioxide within the gas composition of the outside air has a partial pressure of approximately 0.3 mmHg.
Clause 10: The biological liquid collection device of any of Clauses 1-9, wherein the collection module includes a first end having a sample introduction opening, a second end having a sample dispensing opening, a passageway extending between the sample introduction opening and the sample dispensing opening, and a porous plug covering the second end of a housing.
Clause 11: The biological liquid collection device of Clause 10, wherein a closure is configured to close the sample introduction opening in the collection module and wherein the closure comprises a pierceable self-sealing stopper.
Clause 12: The biological liquid collection device of Clause 10 or Clause 11, wherein the porous plug is adapted to allow air to pass from the passageway of the collection module while preventing the biological liquid sample to pass therethrough.
Clause 13: The biological liquid collection device of any of Clauses 1-12, wherein, when the evacuated container is one of a 1 mL 13×75 tube, 2 mL 13×75 tube, 2.5 mL 13×75 tube, 3.5 mL 13×75 tube, and 4 mL 13×75 tube, the evacuated container has a shelf life of at least 10 months, 24 months, 24 months, 12 months, and 21 months, respectively.
Clause 14: The biological liquid collection device of any of Clauses 1-13, wherein the gas composition in the evacuated container further comprises argon.
Clause 15: The biological liquid collection device of any of Clauses 1-14, wherein the gas composition within the evacuated container comprises oxygen, nitrogen, and a third gas such as Argon with a similar permeation rate to nitrogen.
Clause 16: The biological liquid collection device of Clause 15, wherein the oxygen in the gas composition located within the evacuated container has a partial pressure that is equal to the partial pressure of atmospheric oxygen outside of the evacuated container, and the Argon in the gas composition located within the evacuated container has a partial pressure that is greater than the partial pressure of atmospheric Argon outside of the evacuated container.
Clause 17: The biological liquid collection device of Clause 1, wherein the evacuated container is a partial draw tube.
Clause 18: A biological liquid collection device comprising: a collection module for receiving a biological liquid sample; an evacuated container containing the collection module therein; and a closure for closing an open end of the evacuated container, wherein the evacuated container comprises a gas composition that has an enriched oxygen content having a partial pressure substantially greater than a partial pressure of oxygen in air at atmospheric pressure of 760 mmHg external to an inner cavity of the evacuated container.
Clause 19: The biological liquid collection device of Clause 18, wherein the evacuated container has a partial pressure of approximately 300 mmHg and wherein the partial pressure of oxygen within the evacuated container is approximately 160 mmHg.
Clause 20: The biological liquid collection device of Clause 19, wherein the gas composition includes carbon dioxide and nitrogen, and wherein the partial pressure of carbon dioxide within the evacuated container is approximately 0.3 mmHg and the partial pressure of nitrogen within the evacuated container is approximately 140 mmHg.
Clause 21: The biological liquid collection device of any of Clauses 18-20, wherein the evacuated container has a partial pressure of approximately 300 mmHg and wherein the partial pressure of oxygen within the evacuated container is greater than 160 mmHg.
Clause 22: The biological liquid collection device of any of Clauses 18-21, wherein the gas composition comprises approximately 75% oxygen.
Clause 23: The biological liquid collection device of Clause 22, wherein the gas composition further comprises approximately 23% nitrogen and approximately 0.1% carbon dioxide.
Clause 24: A method of making an atmospheric balanced fluid collection device comprising: providing a container having an open end and a closed end, said container defining a chamber; drawing a vacuum within the container to remove at least some gas from within the chamber; back purging the chamber with a gas composition that is proportioned greater than a gas composition of atmosphere outside of an evacuated container, wherein the back purging of the chamber is conducted until reaching a predetermined vacuum pressure within the container; drawing a further vacuum within the container to remove at least some of the gas from within the chamber; back purging the chamber with a further gas composition that is proportioned greater than the gas composition of the atmosphere outside of the evacuated container, wherein the back purging of the chamber is conducted until reaching the predetermined vacuum pressure within the container; and closing the open end of the container.
Clause 25: The method of Clause 24, wherein the predetermined vacuum pressure within the container is 300 mmHg and the gas composition comprises approximately 75% oxygen having a partial pressure of approximately 160 mmHg.
Clause 26: The method of Clause 24 or Clause 25, further including placing a fluid collection module within the container, wherein the fluid collection module comprises a first end having a sample introduction opening, a second end having a sample dispensing opening, a passageway extending between the sample introduction opening and the sample dispensing opening, and a porous plug covering the second end of a housing, said porous plug being adapted to allow air to pass from the passageway of the collection module while preventing a biological liquid sample to pass therethrough.
Clause 27: The method of any of Clauses 24-26, wherein, when the evacuated container is one of a 1 mL 13×75 tube, 2 mL 13×75 tube, 2.5 mL 13×75 tube, 3.5 mL 13×75 tube, and 4 mL 13×75 tube, the evacuated container has a shelf life of at least 10 months, 24 months, 24 months, 12 months, and 21 months, respectively.
Clause 28: A biological liquid collection device assembly, comprising: an evacuated tube for receiving a biological liquid sample; and a barrier packaging, said barrier packaging containing the evacuated tube therein, wherein the barrier packaging comprises a gas composition with select partial pressure of a targeted gas that is substantially greater than a targeted gas partial pressure of atmosphere external to the barrier packaging.
Clause 29: The biological liquid collection device assembly of Clause 28, wherein the evacuated tube also comprises a gas composition with select partial pressure of a targeted gas that is substantially greater than a targeted gas partial pressure of atmosphere external to the barrier packaging.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a front perspective view of a biological liquid collection device having a collection module disposed within an outer housing in accordance with an aspect of the present disclosure;

FIG. 2 is a partial cross-sectional side view of the biological liquid collection device of FIG. 1 in accordance with an aspect of the present disclosure;

FIGS. 3A and 3B are enlarged partial cross-sectional side views of FIGS. 1 and 2 showing the porous plug closing the liquid collection chamber in accordance with an aspect of the present disclosure;

FIGS. 4A and 4B are schematic diagrams illustrating blood gas vacuum bias using a standard vacuum process in a conventional Vacutainer® tube in accordance with principles known in the art;

FIG. 5 is a schematic illustration of adjusting a pressure in a container according to one aspect of the present disclosure;

FIG. 6 is a schematic illustration depicting the flow of oxygen relative to a container of the present disclosure;

FIG. 7 is a graph showing tube pressure versus time of an oxygen backfilled non-gelled tube in accordance with the disclosed invention, as well as a non-backfilled tube;

FIG. 8 is a graph showing tube pressure versus time of an oxygen backfilled gelled tube in accordance with the disclosed invention, as well as a non-backfilled tube;

FIG. 9 is a schematic illustration depicting the flow of oxygen and argon relative to a container of the present disclosure; and

FIG. 10 is a schematic illustration of an evacuated tube with a barrier packaging according to one non-limiting embodiment or aspect of the present disclosure.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

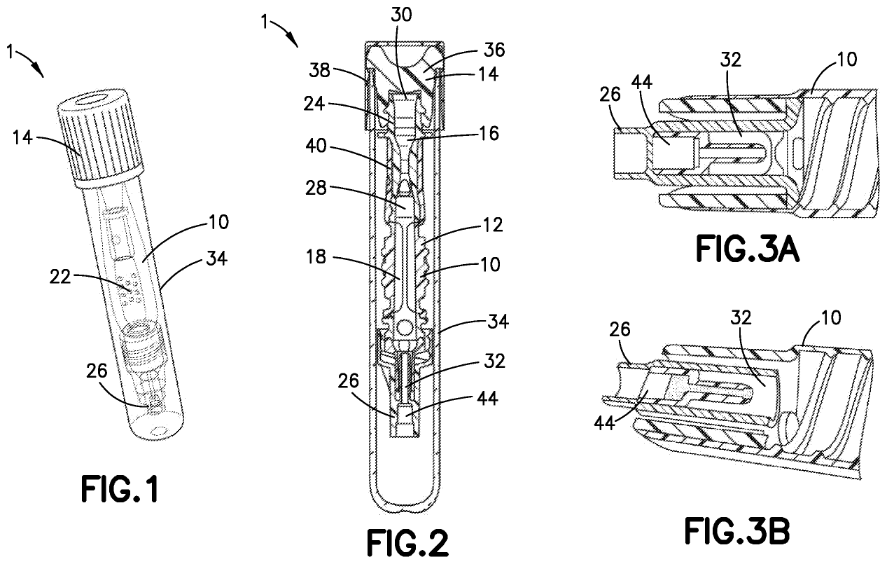

Reference is made to FIGS. 1 and 2, which show a biological liquid collection device, generally indicated as 1, having a collection module 10 disposed within an outer housing or evacuated container 34 in accordance with an aspect of the present disclosure. The collection module 10 is adapted to receive a biological liquid sample, such as a blood sample, and includes a housing 12, a closure 14, a mixing chamber 16, a holding chamber 18, a cap 26, as shown in FIG. 2, and an activation member 22.

In one embodiment, the housing 12 includes a first end 24, a second end 26, and a passageway 28 extending therebetween and providing fluid communication between the first end 24 and the second end 26 of the housing 12. The passageway 28 has a sample introduction opening 30 at the first end 24 of the housing 12 and a sample dispensing opening 32 at the second end 26 of the housing 12. The mixing chamber 16 and the holding chamber 18 are provided in fluid communication with the passageway 28. The mixing chamber 16 and the holding chamber 18 are positioned such that a biological fluid sample, such as a blood sample, introduced into the sample introduction opening 30 of the passageway 28 will first pass through the mixing chamber 16 and subsequently pass into the holding chamber 18, prior to reaching the sample dispensing opening 32 of the passageway 28. In this way, the blood sample may be mixed with an anticoagulant or other additive provided within the mixing chamber 16 before the stabilized sample is received and stored within the holding chamber 18.

The mixing chamber 16 allows for passive mixing of the blood sample with an anticoagulant or another additive, such as a blood stabilizer, as the blood sample flows through the passageway 28. The internal portion of the mixing chamber 16 may have any suitable structure or form as long as it provides for the mixing of the blood sample with an anticoagulant or another additive as the blood sample passes through the passageway 28. The mixing chamber 16 may include a dry anticoagulant, such as Heparin or EDTA, deposited on or within the mixing chamber 16. The mixing chamber 16 may, for example, include an open cell foam containing dry anticoagulant dispersed within the cells of the open cell foam to promote the effectiveness of the flow-through mixing and anticoagulant uptake.

After passing through the mixing chamber 16, the blood sample may be directed to the holding chamber 18. The holding chamber 18 may take any suitable shape and size to store a sufficient volume of blood necessary for the desired testing, for example 500 μl or less. In the embodiment shown in FIGS. 1 and 2, the holding chamber 18 is defined by a portion of the housing 12 in combination with an elastic sleeve 40 secured about the exterior of the housing 12. The elastic sleeve 40 may be made of any material that is flexible, deformable, and capable of providing a fluid tight seal with the housing 12, including, but not limited to, natural or synthetic rubber, and other suitable elastomeric materials.

With continuing reference to FIGS. 1 and 2, and with further reference to FIGS. 3A and 3B, a porous or vented plug 44 is disposed at the second end 26 of the housing 12 and plugs the sample dispensing opening 32 of the passageway 28. The construction of the vented plug 44 allows air to pass therethrough and out of the collection module 10 while preventing the blood sample from passing therethrough and may include a hydrophobic filter. The vented plug 44 has selective air passing resistance that may be used to finely control the filling rate of the passageway 28. By varying the porosity of the plug, the velocity of the air flow out of the plug 44, and thus the velocity of the blood sample flow into the collection module 10, may be controlled. If the blood sample flow velocity into the collection module 10 is too fast, hemolysis may occur. If the blood sample flow velocity into the collection module 10 is too slow, sample collection time may be excessive.

A closure 14 is engaged with the first end 24 of the housing 12 to seal the passageway 28. The closure 14 allows for introduction of a blood sample into the passageway 28 of the housing 12 and may include a pierceable self-sealing stopper 36 with an outer shield 38 such as a Hemogard™ cap commercially available from Becton, Dickinson and Company. The closure 14 also secures to the outer housing or evacuated container 34. It may be appreciated that the evacuated container 34 can be any well-known vacuum containing blood collection tube, such as a Vacutainer® blood collection tube commercially available from Becton, Dickinson and Company.

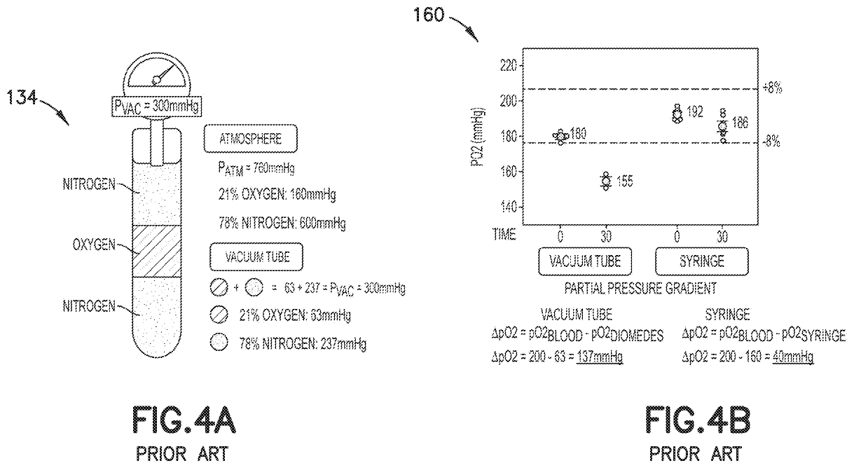

Reference is now made to FIGS. 4A and 4B, which schematically illustrate blood gas vacuum bias using a standard vacuum process in a conventional or prior art evacuated container 200, such as a Vacutainer® container, in accordance with principles known in the art. When blood fills a conventional evacuated container 200, the gas composition, dissolved and bound to hemoglobin in the blood (O₂, N₂, CO₂), is exposed to a gas mixture in the tube where each respective gas mixture component has its own partial pressure. The total pressure (P) in the container 200 is the sum of the partial pressures (P) of each individual gas (P_{tube}=PO₂+PCO₂+PN₂), as demonstrated by Dalton's law of partial pressures. This fundamental property of gases dictates a traditional tube vacuum pressure of 300 mmHg using an atmospheric gas composition (21% O₂, 0.04% CO₂ and 78% N₂) and will result in partial pressures of 63, 12, and 237 mmHg, respectively. The internal pressure of the tube is defined by an internal volume of the tube and a desired draw volume of the tube (e.g., 1 mL, 2 mL, etc.). In comparison, normal atmospheric gas composition has an oxygen partial pressure of 160 mmHg at atmospheric pressure, 760 mmHg at sea level. As indicated by graph 160, as shown in FIG. 4B, the standard vacuum process creates an environment that exposes blood to a larger partial pressure gradient (ΔP) for both oxygen and carbon dioxide in a conventional evacuated container 200 in comparison to a syringe that can then lead to blood gas bias. Henry's law states that the amount of dissolved gas is proportional to its partial pressure in the gas phase. This equilibrium constant shows that the partial pressure of blood gases are directly proportional to the partial pressure of the gas in the tube. As a result, gases in the conventional container 200 as discussed above and shown in FIG. 4A, will come out of solution (blood), as determined by the equilibrium between the undissolved gas in the evacuated container and the gas dissolved in the blood.

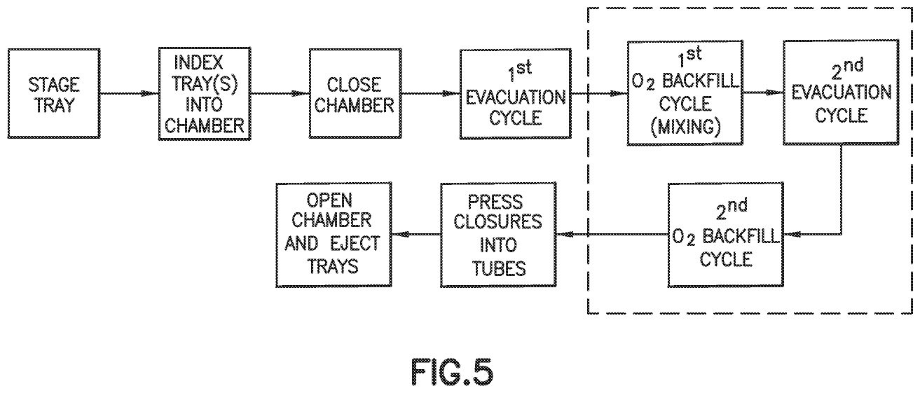

Reference is now made to FIG. 5, which schematically illustrates the liquid evacuated container 34, and method of preparing the evacuated tube 34 in accordance with the present disclosure, wherein the evacuated container 34, which contains the collection module 10, comprises a gas composition that has a pressure that is greater than the pressure of the gas composition of the atmosphere outside of the evacuated container 34. In one non-limiting embodiment or aspect of this disclosure, the term “outside” may be understood to mean an area or position external to an inner cavity of the evacuated container 34. In another example, the gas composition has a pressure that is equal to or matches the pressure of the gas composition of the atmosphere outside of the evacuated container 34 (~160 mmHg). The proposed device adjusts the fundamental partial pressure composition of oxygen, O₂, and carbon dioxide, CO₂, within the vacuum chamber relative to that of the atmospheric conditions, to provide a blood gas sample equivalent to a standard arterial blood gas ABG syringe (current standard of care). In one non-limiting embodiment or aspect of the disclosure, the O₂ may be considered a targeted gas since the partial pressure of O₂ is being adjusted relative to the partial pressure of the O₂ in the atmosphere external to the inner cavity of the evacuated container 34. In other embodiments of the present disclosure, the targeted gas may be different from O₂ or in addition to the O², such as the Argon gas discussed below. In one embodiment or aspect, the targeted gas is understood to be the particular gas of the gas composition that is to be adjusted so as to reduce or eliminate the same targeted gas external from the evacuated container 34 seeping into the evacuated container 34. This was accomplished by developing a vacuum assembly procedure where a high vacuum is pulled and then oxygen, O₂, and carbon dioxide, CO₂, are backfilled into the chamber until achieving the desired final vacuum level and partial pressures of O₂ and CO₂.

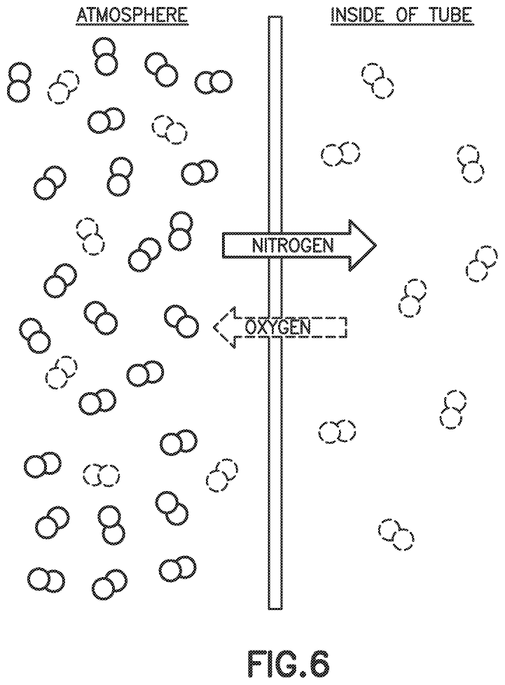

With reference to FIGS. 5 and 6, the presently disclosed device and method results in the collection of blood samples into a vacuum chamber or into the evacuated container 34 where blood is exposed to an increased pressure as compared to the atmospheric partial pressure of oxygen (PO₂) and partial pressure of carbon dioxide (PCO₂) levels found in a standard arterial blood gas syringe, which exposes the blood sample to normal atmospheric air, and its respective PO₂ and PCO₂ levels, as shown in the graph of FIG. 4B. With continuing reference to FIG. 5, the method for obtaining the pressure adjusted container 34 of the present disclosure is achieved by starting with a container which is at atmospheric pressure, 760 mmHg, comprising a composition of approximately 21% oxygen, O₂, and 79% nitrogen, N₂, having a partial pressure of nitrogen (PN₂) of approximately 160 mmHg, and a partial pressure of oxygen PO₂ of approximately 600 mmHg. Next, a high vacuum is pulled from within the tube to where most of the gas is removed from the chamber 135 so that the tube has a total pressure of approximately 20 mmHg and the composition of the tube is approximately 21% oxygen, O₂ having a partial pressure PO₂ of approximately 37 mmHg, and approximately 79% nitrogen, N₂ having a partial pressure PN₂ of approximately 140 mmHg. In a subsequent step, another high vacuum is pulled from within the tube to where most of the gas is again removed from the chamber 135. In a final step, the tube is back purged with a deliberately proportioned gas composition of O2, N₂, and CO₂ until reaching the desired vacuum level of approximately 300 mmHg that is greater than atmospheric partial pressures of O₂ and CO₂ forming the pressure adjusted evacuated tube 34, of the disclosure, as shown in FIG. 5, wherein the composition of the tube is approximately 30%-100% oxygen and up to 70% nitrogen, and the partial pressure of oxygen PO₂ is approximately 160 mmHg, the partial pressure of nitrogen PN₂ is approximately 140 mmHg, and the partial pressure of carbon dioxide PCO 2 is approximately 0.3 mmHg. In one example of the present disclosure, the composition of the tube is approximately greater than 50% oxygen and less than 50% nitrogen. In another example of the present disclosure, the composition of the tube may be 100% oxygen. It may be appreciated that the tube can be back purged such that the partial pressure of oxygen within the evacuated container is greater than 160 mmHg. In another example, an oxygen barrier may be provided in the body of the evacuated tube to assist in preventing gas permeation.

In another embodiment or aspect of the present disclosure, the PO₂ (or another gas) in the tube may be deliberately above PO₂ (or another gas) in the atmosphere (e.g. hypersaturation within the tube). This could also be used to allow to increase draw volume over time, if so desired. This is potentially useful in tubes with higher internal pressure/internal volume ratios (e.g. a partial draw tube). Use of this embodiment further extends the product shelf-life (beyond what is already discussed). In one non-limiting embodiment or aspect of the present disclosure, a partial draw tube is understood to be a blood collection tube that is smaller (in one example, approximately 3.0 mL or less) than a standard (in one example, approximately 4.5 mL) draw tube. In some situations, a partial draw tube is used when less blood is needed for testing and analysis. Partial draw tubes may be used for a variety of uses, including blood donor screening and infectious disease testing, plasma determinations, serum determinations, hematology determinations, immunohematology, and routine coagulation studies.

By increasing the oxygen content in the tube, the oxygen gradient within the atmosphere is reduced to slow down or eliminate oxygen permeation into the tube. Due to the increased oxygen content in the tube, atmospheric oxygen is largely reduced or prevented from permeating into the tube since the oxygen content in the tube blocks this permeation.

Pressure-adjusted partial pressure PO₂ and PCO₂ vacuum tube architecture enables stable blood gas levels during blood vacuum draws using conventional blood collection sets based on the typical evacuated container systems.

Vacuum shelf-life loss in the evacuated containers 134 of the prior art is due to gas permeation through the plastic tube, which is driven by the atmospheric and vacuum partial pressure gradient at the plastic barrier. Nitrogen contributes the least to vacuum loss as the permeation factor for oxygen is an order of magnitude higher in polyethylene terephthalate (PET), a plastic primarily used in typical evacuated tubes. The above-described pressure-adjusted vacuum tube architecture provides a superior vacuum shelf-life, as the increased PO₂ and PCO₂ gradients in the tube are not susceptible to gas permeation. This is due to the fact that by design there is an increased difference in PO₂ and PCO₂ pressures inside and outside the container 134 of the prior art. For example, when the total pressure of atmospheric air outside of the evacuated container is 760 mmHg, the oxygen within the gas composition of the outside air has a partial pressure of approximately 160 mmHg, and the carbon dioxide within the gas composition of the outside air has a partial pressure of approximately 0.3 mmHg. In the pressure-adjusted evacuated tube 34 of the present disclosure, the oxygen within the gas composition within the tube also has a partial pressure of approximately 160 mmHg, and the carbon dioxide within the gas composition within the tube has a partial pressure of approximately 0.3 mmHg, but the composition inside the tube has an increased percentage of oxygen. Because the oxygen percentage of the composition is increased inside the tube, there is no pressure exchange and no resultant vacuum loss from the O₂ and CO₂. The difference in partial pressure of nitrogen N₂ within and outside of the tube can be significantly different, i.e., the partial pressure of nitrogen PN₂ within the tube is approximately 140 mmHg and the partial pressure of nitrogen PN₂ within the atmosphere outside of the tube is approximately 593 mmHg. This difference in partial pressure can result in a slight increase in the vacuum pressure within the tube due to nitrogen N₂ permeation into the tube because nitrogen has approximately 10× lower permeability vs. oxygen. It is noted herein that the pressure-adjusted compositions as described could be useful in increasing the shelf-life of any conventional specimen collection container. For example, this pressure adjusting technique could be useful for prolonging the shelf-life of plastic blood collection containers, including any kind of evacuated tube. Although this application has particular applicability to arterial blood gas applications, the pressure adjusting methodologies described herein can be utilized for any evacuated plastic container. In addition, it is anticipated herein that the pressure adjusting methodologies identified herein may be suitably used in venous or other blood collection applications.

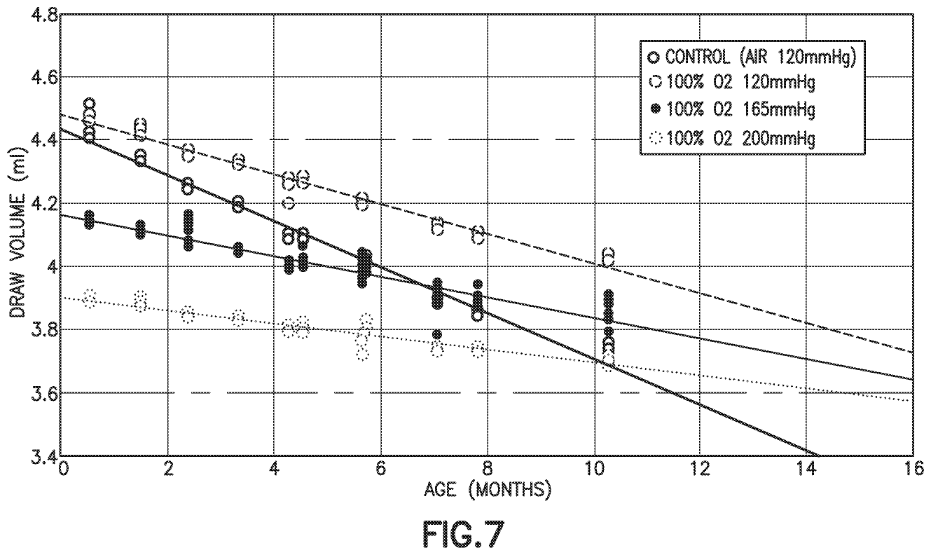

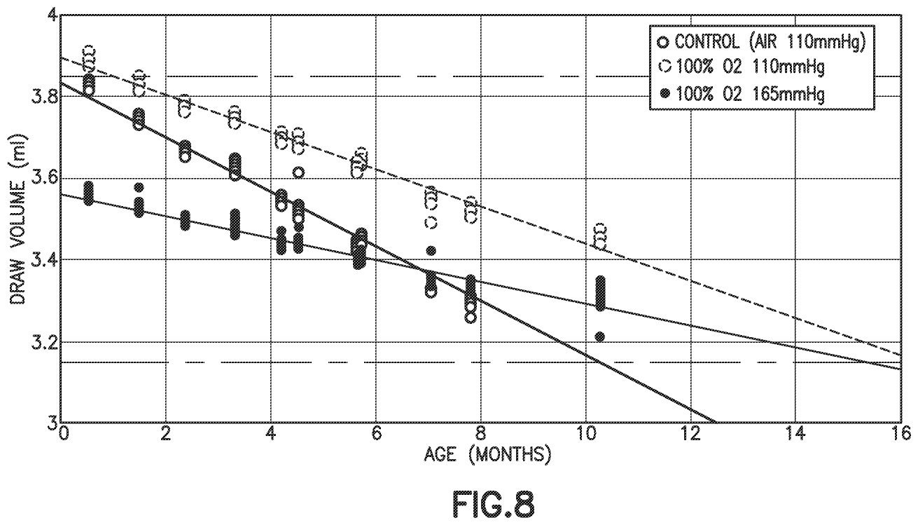

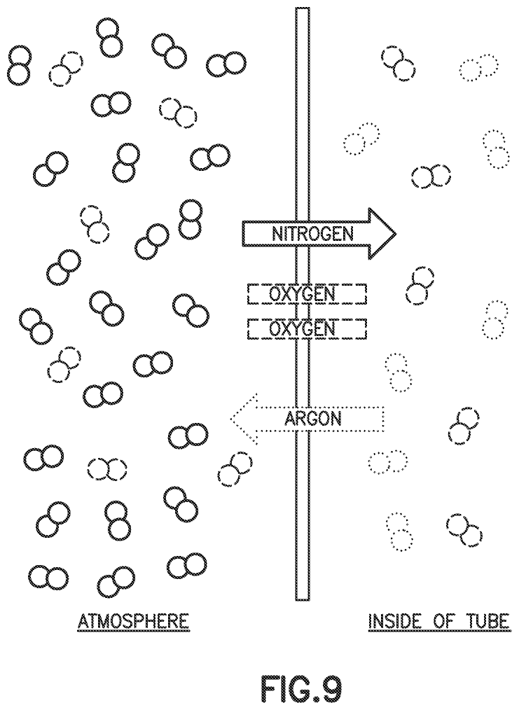

FIGS. 7 and 8 illustrate different graphs of draw volume as a function of time due to the permeability of the tube being tested. As shown in each of the plots, O₂ backfilled tubes have been found to take significantly longer to reach a threshold (e.g., a critical 20% threshold wherein the tube can still draw within 20% of the volume that it drew when it was first evacuated, referred to herein as “shelf life”) than counterpart non-backfilled tubes. As a result, this corresponds to a significantly improved shelf life for the O₂ backfilled tubes. FIG. 6 illustrates validation results achieved with a 13×75 mL non-gelled tube. FIG. 7 illustrates validation results achieved with a 13×75 mL gelled tube. Specifically, when the evacuated container is one of a 1 mL 13×75 tube, 2 mL 13×75 mL tube, 2.5 mL 13×75 tube, 3.5 mL 13×75 tube, and 4 mL 13×75 tube, the evacuated container has a shelf life of at least 10 months, 24 months, 24 months, 12 months, and 21 months, respectively. These results show that this oxygen pressure adjusted method increases the shelf life of the containers by at least six months.

It can be appreciated that patients exposed to hyperoxia conditions over a prolonged period can experience a higher than normal partial pressure of oxygen that can exceed 500 mmHg. Under these conditions, gas is forced to dissolve in an unbound state in the plasma of blood while a smaller portion is still bound to hemoglobin. During blood gas analysis, these samples can exhibit higher bias levels within the typical 15 minute turn-around time, as oxygen in plasma has a high dissolution gas exchange rate combined with the partial pressure gradient when blood is exposed to atmosphere. Hyperoxia (relative to atmospheric PO₂ and PCO₂) PO₂ and PCO₂ levels could be used in the vacuum tube architecture to further improve blood gas stability for an oxygen therapy product that isn't susceptible to bias at extremes. This is feasible for ABG applications as the device design doesn't have a high enough surface area required to positively bias blood gas levels. This would never be possible in a classic ABG syringe.

As shown in FIG. 8, according to another embodiment or example of the present disclosure, the method of adjusting the composition in the tube may also include the introduction of a third gas, besides oxygen and nitrogen, to provide the potential for an even longer shelf life benefit for the evacuated tube. By using this method, the pressure in the tube is maintained at nearly constant levels for a significant portion of the shelf life of the tube. Using this method, the oxygen in the tube is matched to the oxygen content in the atmosphere to effectively eliminate oxygen permeation. A third gas is also introduced into the evacuated tube to counteract nitrogen permeation from atmosphere into the tube. In one example of the present disclosure, the third gas may be argon. Argon has a similar permeation rate to nitrogen and nearly balances nitrogen permeation from atmosphere into the tube. It should also be understood that there are scenarios where deliberate and purposeful combinations of gases, which combinations of more than three gases, could be used (e.g. volatile gas/gasses coming from component(s) within the evacuated space, an intentional chemical reaction to release gas/gases, or other) to achieve the same desired outcome (e.g. a gaseous substance at an intentional partial pressure with permeability similar to nitrogen).

According to one non-limiting embodiment or aspect of the present disclosure, a method of controlling a vacuum composition of an evacuated blood collection container may be used to improve device blood gas test performance. In this example, an evacuated tube may have a vacuum with a controlled and optimized oxygen pressure (pO2) to improve device blood gas performance. The controlled vacuum composition may be achieved by pulling vacuum in the tube and backfilling the tube with a gas mixture during the tube evacuation process. In one example, the controlled vacuum composition may include a tube oxygen pressure pO2 of approximately 70 mmHg. It is to be understood that 70 mmHg is just one pressure reading that can be used for optimized device oxygen pressure to improve blood gas test performance. The optimized device oxygen pressure may be greater or lower than 70 mmHg as needed based on the type of device being used. In one example, the gas mixture backfilled into the tube may include at least one of the following: nitrogen, oxygen, or a combination of nitrogen and oxygen. Several advantages are realized using this method. In particular, vacuum-based devices, such as a vacutainer, are generally not recommended for blood gas tests because of head space after blood sample collection. The head space (or air bubble) can induce erroneous results by oxygen exchange with the blood sample. However, the optimized oxygen pressure in the tube described above significantly improves the oxygen pressure (pO2) blood gas performance and extends the pO2 test range. However, the controlled vacuum composition as a fresh product should be maintained over the shelf life of the product. In one example, the tube O2 pressure can be controlled to be lower than atmospheric pressure (pO2) but still higher than a regular evacuated tube. This control will provide balanced O2 pressure in the device headspace after sample collection compared to the pO2 in the blood sample, which in turn reduces O2 transfer from the headspace to the sample or vice versa during the turnaround time before testing the blood sample.

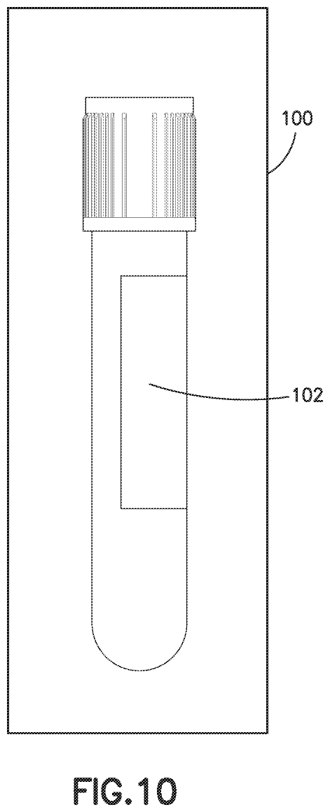

With reference to FIG. 10, according to a non-limiting embodiment or aspect of the present disclosure, a barrier packaging 100 with a controlled oxygen pressure may be used to extend a product shelf life of an evacuated tube 102. In this example, the packaging 100 may have a controlled oxygen pressure that matches the controlled oxygen pressure of the evacuated tube 102. In some examples, the packaging 100 may be a foil pouch, a blister pack, a foil film shelf pack, an oxygen barrier shrink wrap, or any other packaging used to store an evacuated tube 102 or fluid container. By matching the controlled oxygen pressure of the packaging 100 with the controlled oxygen pressure of the evacuated tube 102, oxygen permeation from the evacuated tube 102 is reduced and the product shelf life of the evacuated tube 102 is extended. The oxygen pressure of the packaging 100 may be controlled by pulling vacuum and/or backfilling the packaging with gas, such as nitrogen or oxygen, during the packaging process to match the oxygen pressure of the evacuated tube 102. The matched oxygen pressure in the packaging 100 reduces oxygen permeation and extends the shelf life of the evacuated tube 102.

Evacuated blood collection tubes have typically much lower oxygen pressure (e.g. ~0-20 mmHg) compared to atmosphere (~160 mmHg). This pressure difference generates oxygen permeation through the tube wall of the blood collection tubes, which induces vacuum loss and limits the product shelf life of the blood collection tube. The oxygen barrier packaging 100 with controlled oxygen pressure can extend the product shelf life without changing the tube design and/or material by reducing oxygen permeation. The controlled oxygen pressure in the packaging 100 can accommodate semi-barrier packaging material.

In one non-limiting embodiment or aspect of the present disclosure, an evacuated tube may have a vacuum with a controlled and optimized oxygen pressure (pO2) to improve device blood gas performance. The controlled vacuum composition may be achieved by pulling vacuum in the tube and backfilling the tube with a gas mixture during the tube evacuation process. In addition to the controlled vacuum composition, a barrier packaging for the evacuated tube may have a controlled oxygen pressure that matches the controlled oxygen pressure of the evacuated tube. Therefore, using this process, the shelf life of the evacuated tube is extended and the oxygen permeation for the evacuated tube is reduced by using a combination of the controlled vacuum composition in the evacuated tube and the barrier packaging with the controlled oxygen pressure.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure that are known or customarily practiced in the art to which this disclosure pertains and which fall within the limits of the appended claims.

The invention claimed is:

1. A biological liquid collection device, comprising:
a collection module for receiving a biological liquid sample;
an evacuated container having an open end and a closed end, said evacuated container containing the collection module therein; and
a closure for closing the open end of the evacuated container, wherein the evacuated container comprises a gas composition with select partial pressure of a targeted gas that is substantially greater than a targeted gas partial pressure of atmosphere external to an internal cavity of the evacuated container.

2. The biological liquid collection device of claim 1, wherein the evacuated container comprises the gas composition with select partial pressures of targeted gasses that are greater than the targeted gas partial pressures of the atmosphere outside of the evacuated container.

3. The biological liquid collection device of claim 1, wherein the gas composition within the evacuated container comprises oxygen, carbon dioxide, and nitrogen.

4. The biological liquid collection device of claim 3, wherein the oxygen in the gas composition located within the evacuated container has a partial pressure that is greater than a partial pressure of atmospheric oxygen outside of the evacuated container.

5. The biological liquid collection device of claim 3, wherein the carbon dioxide in the gas composition located within the evacuated container has a partial pressure that is substantially equal to a partial pressure of atmospheric carbon dioxide outside of the evacuated container.

6. The biological liquid collection device of claim 3, wherein the gas composition comprises approximately 75% oxygen, approximately 23% nitrogen, and approximately 0.1% carbon dioxide.

7. The biological liquid collection device of claim 6, wherein the evacuated container has a total pressure of 300 mmHg and wherein the oxygen within the gas composition in the evacuated container has a partial pressure of approximately 160 mmHg.

8. The biological liquid collection device of claim 7, wherein the evacuated container has a total pressure of 300 mmHg and wherein the carbon dioxide within the gas composition in the evacuated container has a partial pressure of approximately 0.3 mmHg.

9. The biological liquid collection device of claim 8, wherein the oxygen within the gas composition of outside air has a partial pressure of approximately 160 mmHg and the carbon dioxide within the gas composition of the outside air has a partial pressure of approximately 0.3 mmHg.

10. The biological liquid collection device of claim 1, wherein the collection module includes a first end having a sample introduction opening, a second end having a sample dispensing opening, a passageway extending between the sample introduction opening and the sample dispensing opening, and a porous plug covering the second end of a housing.

11. The biological liquid collection device of claim 10, wherein a closure is configured to close the sample introduction opening in the collection module and wherein the closure comprises a pierceable self-sealing stopper.

12. The biological liquid collection device of claim 10, wherein the porous plug is adapted to allow air to pass from the passageway of the collection module while preventing the biological liquid sample to pass therethrough.

13. The biological liquid collection device of claim 1, wherein, when the evacuated container is one of a 1 mL 13×75 tube, 2 mL 13×75 tube, 2.5 mL 13×75 tube, 3.5 mL 13×75 tube, and 4 mL 13×75 tube, the evacuated container has a shelf life of at least 10 months, 24 months, 24 months, 12 months, and 21 months, respectively.

14. The biological liquid collection device of claim 1, wherein the gas composition in the evacuated container further comprises argon.

15. The biological liquid collection device of claim 1, wherein the gas composition within the evacuated container comprises oxygen, nitrogen, and a third gas such as Argon with a similar permeation rate to nitrogen.

16. The biological liquid collection device of claim 15, wherein the oxygen in the gas composition located within the evacuated container has a partial pressure that is equal to the partial pressure of atmospheric oxygen outside of the evacuated container, and the Argon in the gas composition located within the evacuated container has a partial pressure that is greater than the partial pressure of atmospheric Argon outside of the evacuated container.

17. The biological liquid collection device of claim 1, wherein the evacuated container is a partial draw tube.

18. The biological liquid collection device of claim 3, wherein the oxygen in the gas composition located within the evacuated container has a partial pressure that is lower than a partial pressure of atmospheric oxygen outside of the evacuated container

19. A biological liquid collection device comprising:
a collection module for receiving a biological liquid sample;
an evacuated container containing the collection module therein; and
a closure for closing an open end of the evacuated container, wherein the evacuated container comprises a gas composition that has an enriched oxygen content having a partial pressure substantially greater than a partial pressure of oxygen in air at atmospheric pressure of 760 mmHg external to an inner cavity of the evacuated container.

20. The biological liquid collection device of claim 19, wherein the evacuated container has a partial pressure of approximately 300 mmHg and wherein the partial pressure of oxygen within the evacuated container is approximately 160 mmHg.

21. The biological liquid collection device of claim 20, wherein the gas composition includes carbon dioxide and nitrogen, and wherein the partial pressure of carbon dioxide within the evacuated container is approximately 0.3 mmHg and the partial pressure of nitrogen within the evacuated container is approximately 140 mmHg.

22. The biological liquid collection device of claim 19, wherein the evacuated container has a partial pressure of approximately 300 mmHg and wherein the partial pressure of oxygen within the evacuated container is greater than 160 mmHg.

23. The biological liquid collection device of claim 19, wherein the gas composition comprises approximately 75% oxygen.

24. The biological liquid collection device of claim 23, wherein the gas composition further comprises approximately 23% nitrogen and approximately 0.1% carbon dioxide.

25. A method of making an atmospheric balanced fluid collection device comprising:
providing a container having an open end and a closed end, said container defining a chamber;
drawing a vacuum within the container to remove at least some gas from within the chamber;
back purging the chamber with a gas composition that is proportioned greater than a gas composition of atmosphere outside of an evacuated container, wherein the back purging of the chamber is conducted until reaching a predetermined vacuum pressure within the container;
drawing a further vacuum within the container to remove at least some of the gas from within the chamber;
back purging the chamber with a further gas composition that is proportioned greater than the gas composition of the atmosphere outside of the evacuated container, wherein the back purging of the chamber is conducted until reaching the predetermined vacuum pressure within the container; and
closing the open end of the container.

26. The method of claim 25, wherein the predetermined vacuum pressure within the container is 300 mmHg and the gas composition comprises approximately 75% oxygen having a partial pressure of approximately 160 mmHg.

27. The method of claim 25, further including placing a fluid collection module within the container, wherein the fluid collection module comprises a first end having a sample introduction opening, a second end having a sample dispensing opening, a passageway extending between the sample introduction opening and the sample dispensing opening, and a porous plug covering the second end of a housing, said porous plug being adapted to allow air to pass from the passageway of the collection module while preventing a biological liquid sample to pass therethrough.

28. The method of claim 25, wherein, when the evacuated container is one of a 1 mL 13×75 tube, 2 mL 13×75 tube, 2.5 mL 13×75 tube, 3.5 mL 13×75 tube, and 4 mL 13×75 tube, the evacuated container has a shelf life of at least 10 months, 24 months, 24 months, 12 months, and 21 months, respectively.

29. A biological liquid collection device assembly, comprising:
an evacuated tube for receiving a biological liquid sample; and
a barrier packaging, said barrier packaging containing the evacuated tube therein,
wherein the barrier packaging comprises a gas composition with select partial pressure of a targeted gas that is substantially greater than a targeted gas partial pressure of atmosphere external to the barrier packaging.

30. The biological liquid collection device assembly of claim 29, wherein the evacuated tube also comprises a gas composition with select partial pressure of a targeted gas that is substantially greater than a targeted gas partial pressure of atmosphere external to the barrier packaging.

31. The biological liquid collection device assembly of claim 29, wherein the evacuated tube also comprises a gas composition with select partial pressure of a targeted gas that is substantially lower than a targeted gas partial pressure of atmosphere external to the barrier packaging.

32. The biological liquid collection device assembly of claim 29, wherein the evacuated tube also comprises a gas composition with select partial pressure of a targeted gas that is substantially equal to a targeted gas partial pressure of atmosphere external to the barrier packaging.
